(19) United States
(12) Patent Application Publication
(10) Pub. No.: US 2005/0148064 A1
(43) Pub. Date: Jul. 7, 2005

(54) Microfluid molecular-flow fractionator and bioreactor with integrated active/passive diffusion barrier

(75) Inventors: Mineo Yamakawa, Campbell, CA (US); John Heck, Berkeley, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/748,389

(22) Filed: Dec. 29, 2003

(51) Int. Cl. C12M 1/34

(52) U.S. Cl. 435/287.2; 435/288.5; 435/288.7

(57) ABSTRACT

A microfluidic device and method is disclosed for fractionating and/or trapping selected molecules with a diffusion barrier or porous membrane. The device includes a source fluid flow channel and a target fluid flow channel. The target fluid flow channel and the source fluid flow channel meet at cross-channel area and are in fluid communication with each other. A porous membrane separates the source fluid flow channel from the target fluid flow channel in the cross-channel area. A field-force/gradient mechanism may be positioned proximate the porous membrane with or without detection/state monitoring devices.

### BACKGROUND OF THE INVENTION

1. Field of the Invention

This disclosure relates generally to microfluidic devices with diffusion barriers, and more specifically, to microfluidic devices having active/passive porous membrane diffusion barriers for fractionation and/or molecular trapping.

2. Background Information

As the breadth of microchip fabrication technology has continued to expand, an emerging technology associated with miniscule gadgets known as microfluidic devices has taken shape. Microfluidic devices, often comprising miniaturized versions of reservoirs, pumps, valves, filters, mixers, reaction chambers, and a network of capillaries interconnecting the microscale components, are being developed to serve in a variety of deployment scenarios. For example, microfluidic devices may be designed to perform multiple reaction and analysis techniques in one micro-instrument by providing a capability to perform hundreds of operations (e.g. mixing, heating, separating) without manual intervention. In some cases, microfluidic devices may function as detectors for airborne toxins, rapid DNA analyzers for crime-scene investigators, and/or new pharmaceutical testers to expedite drug development.

While the applications of such microfluidic devices and sensing substrates may be virtually boundless, the integration of some microscale components into microfluidic systems has been technically difficult, thereby limiting the range of functions that may be accomplished by a single device or combination of devices. In particular, current microfluidic systems have not adequately integrated a size-separating (or excluding) filter into a microfluidic chip. As such, separations may generally be carried out in external packed porous media or polymer-based nanopore membranes, thereby increasing contamination risks and introducing additional complexity and manual interaction into the performance of an analysis or other technique. Furthermore, sensing substrates have also not been integrated into a chip or the like.

Different methods have been used to separate or fractionate molecules or particles of interest, such as field-flow fractionation (FFF) and split-flow thin fractionation (SPLITT) (both shown in FIG. 1), chromatography with fraction collector, electrophoresis, polymer membrane filtering, etc. These methods may require relatively large sample volumes and consume a significant amount of time to fractionate the samples. In addition, the methods may be limited to specific range of molecular properties, such as size, weight, etc.

What is needed is a device and method for fractionating and/or trapping molecules/particles of interest.

### BRIEF DESCRIPTION OF THE DRAWINGS

In the following detailed description of the invention reference is made to the accompanying drawings which form a part hereof, and in which are shown, by way of illustration, specific embodiments in which the invention may be practiced. These embodiments are described in sufficient detail to enable those skilled in the art to practice the invention. Other embodiments may be utilized, and structural, logical, and electrical changes may be made, without departing from the scope of the present invention.

FIG. 1 shows conventional fractionation methods using split-flow thin fractionation (SPLITT) and field-flow fractionation (FFF).

FIGS. 2a-f are various views of a microfluidic device in accordance with one embodiment of the invention, wherein FIGS. 2a and 2b are exploded isometric views, FIG. 2c is a cross-section view corresponding to section cut 2c-2c, FIG. 2d is a isometric hidden line view, FIG. 2e is an isometric view including a composite section cut, and FIG. 2f is a plan view including section cut 2c-2c.

FIGS. 3a-f show various embodiments of the microfluidic device shown in FIG. 2. FIG. 3a shows fluid flow through the device. FIGS. 3b-c are cross-sectional view of the cross-channel area showing the upper and lower channels and the active/passive diffusion barrier. FIG. 3d shows a microfluidic device with an electric field applied for electrokinetic and electroosmotic reactions. FIG. 3e is a cross-sectional view with the addition of a field force/gradient. FIG. 3f is a cross-sectional view showing molecular trapping.

FIGS. 4a-e are various views of a microfluidic device in accordance with another embodiment of the invention in which an array of porous membrane/sensors are employed, wherein FIG. 4a is an exploded isometric view, FIG. 4b is an assembled isometric view, FIG. 4c is a plan view including section cuts 4d-4d and 4e-4e, FIG. 4d is a cross-section view corresponding to section cut 4d-4d, and FIG. 4e is a cross-section view corresponding to section cut 4e-4e.

FIG. 5 shows the fluid flow of the device shown in FIG. 4.

FIGS. 6a-c depict various views of optical sensing equipment implemented for detecting changes in an optical characteristic of a porous membrane/sensor corresponding to the embodiment of FIGS. 2a-f, wherein volumes internal to the substrate are shown.

FIGS. 7a-c depict various views of optical sensing equipment implemented for detecting changes in an optical characteristic of a porous membrane/sensor corresponding to the embodiment of FIGS. 4a-e wherein volumes internal to the substrate are shown.

FIG. 8 is a schematic diagram illustrating an embodiment of the invention for detecting changes in an electrical characteristic of a porous membrane/sensor.

FIG. 9a is a flow chart illustrating operations that may be used to fabricate a porous membrane in accordance with one embodiment of the invention and FIG. 9b is a flowchart illustrating operations that may be used to fabricate a porous membrane in accordance with another embodiment of the invention.

FIG. 10 shows the fabrication steps of one embodiment of a microfluidic device.

FIG. 11a shows a simplified cross-sectional view of the cross-channel area of the microfluidic device of FIG. 10.

### DETAILED DESCRIPTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of the “a” or “an” are employed to describe elements and components of the invention. This is done merely for convenience and to give a general sense of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Embodiments of a MICROFLUIDIC MOLECULAR-FLOW FRACTIONATOR AND BIOREACTOR WITH INTEGRATED ACTIVE/PASSIVE DIFFUSION BARRIER, and methods for fabricating and using the device are described in detail herein. In the following description, numerous specific details are provided, such as the identification of various system components, to provide an understanding of embodiments of the invention. One skilled in the art will recognize, however, that embodiments of the invention can be practiced without one or more of the specific details, or with other methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more embodiments.

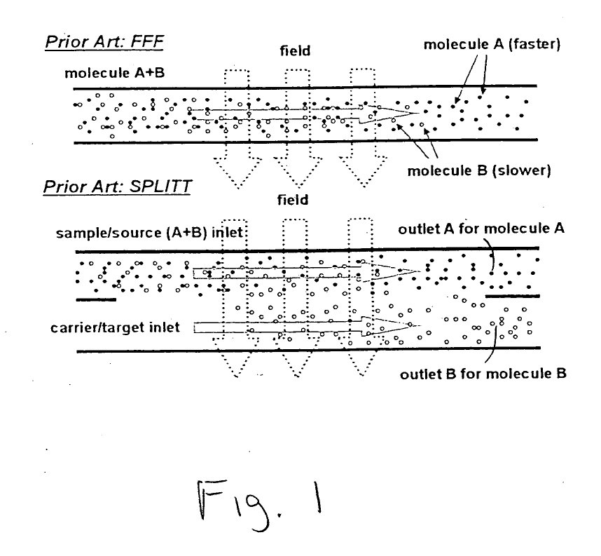

As an overview, embodiments of the invention provide a microfluidic device with at least one porous membrane, for example, a porous-silicon membrane, used as a passive and/or active diffusion barrier between a source (sample) fluid and a target (carrier) fluid, particularly for fractionation and molecular trapping. Fractionation without a barrier has been used in the prior art (see FIG. 1). With the incorporation of the porous membrane as a barrier, the driving force field imposed on the equilibrium phase or transport phase between the two fluid interfaces can influence and/or modulate the functionality of the porous membrane, such as affinity, mobility, charges, magnetic properties, etc. Also, the porous membrane's inherent optical, electrical, acoustic, and/or any other unique properties as a sensor element can be potentially utilized to create a built-in detector for the state of fractionation processes and molecules. Other features of the illustrated embodiments will be apparent to the reader from the foregoing and the appended claims, and as the detailed description and discussion is read in conjunction with the accompanying drawings.

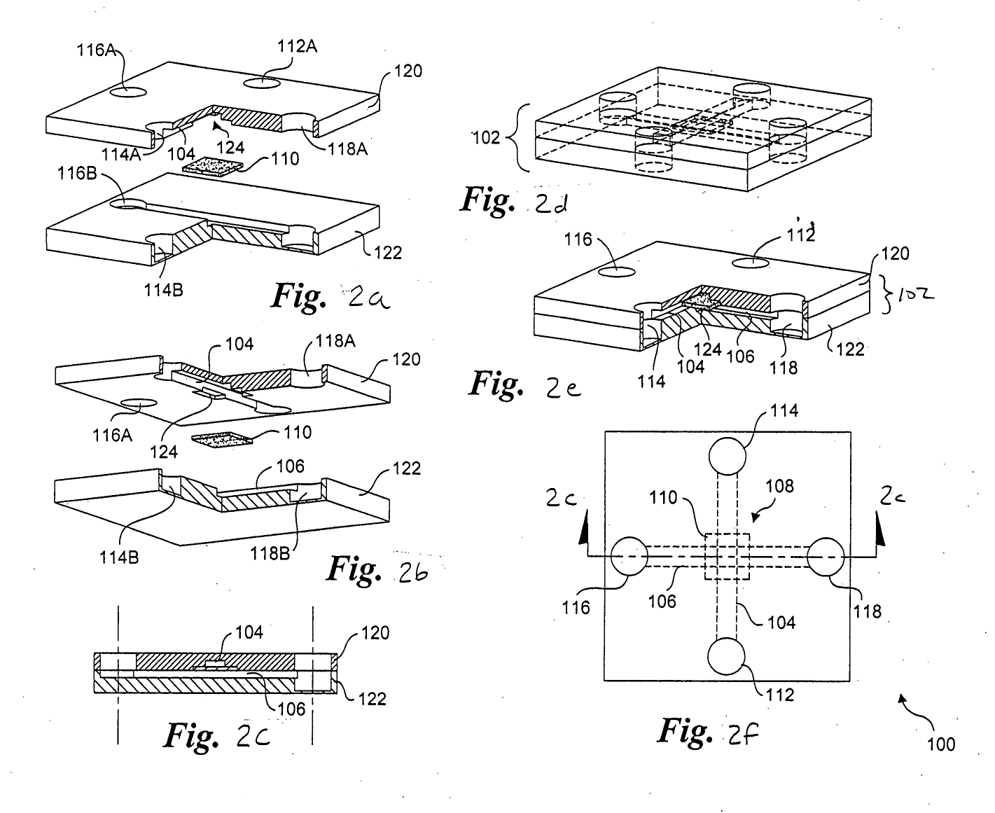

A microfluidic device 100 in accordance with one embodiment of the invention is shown in FIGS. 2a-f. Microfluidic device 100 includes a substrate 102 in which an upper microfluidic channel 104 and lower microfluidic channel and 106 are formed. The upper and lower microfluidic channels 104, 106 are oriented such that the upper channel 104 crosses over the lower channel 106 at a “cross-channel” area 108. A porous membrane 110 is positioned between the upper channel 104 and lower channel 106 proximate to this cross-channel area 108. As described below in further detail, the porous membrane 110 includes a plurality of pores through which molecular portions or particles of interest of some fluids, including liquids and gases, may pass, while restricting passage of other molecules or particles.

In various embodiments, reservoirs may be connected to one or both ends of the upper channel 104 and/or the lower channel 106. For example, in the illustrated embodiment, an input reservoir 112 and output reservoir 114 are connected at respective input and output ends of upper channel 104, while an input reservoir 116 and an output reservoir 118 are connected at respective input and output ends of lower channel 106. In general, it may be desired to have liquid flow through each of the upper and lower channels in a particular direction. In consideration of this, the depth of the output reservoirs may extend below the channel depth. As a result, when fluid is added to the input reservoirs 112, 116, it is caused to flow through the channels to the output reservoirs 114, 118. In place of or in addition to the output reservoirs, respective exit paths for the upper and lower channels may also be provided (not shown).

One embodiment of the substrate 102 is shown in FIG. 2. The substrate 102 includes an upper substrate 120 and a lower substrate 122, which are sandwiched around a porous membrane/sensor 110. The upper microfluidic channel 104 is formed in the upper substrate member 120, while the lower microfluidic channel 106 is formed in the lower substrate 122. Lower portions 116B, 118B and 114B of input reservoir 116 and output reservoirs 118 and 114, may be formed in the lower substrate member 122, while corresponding through holes 112A, 114A, 116A and 118A are defined in the upper substrate member 120.

In the embodiment shown, the porous membrane 110 is sandwiched between the upper 120 and lower 122 substrate members upon assembly. Accordingly, a recess 124 in which the porous membrane 110 will be disposed upon assembly may be formed in either the upper or lower substrate member. For example, in the illustrated embodiment, the recess 124 is defined in upper substrate member 120.

In another embodiment, the porous membrane 110 may be made fabricated as an integral part of the substrate 102. For example, the upper substrate 120 and a lower substrate 122 may be made of silicon. One or both of the silicone layers may be etched, either by electrochemical etching or stain etching, to form a porous silicon (PSi). The porosity, pore size, orientation of the pores, etc, are controlled by the etching conditions (e.g., current, density, etc.) and substrate type and its electrochemical properties.

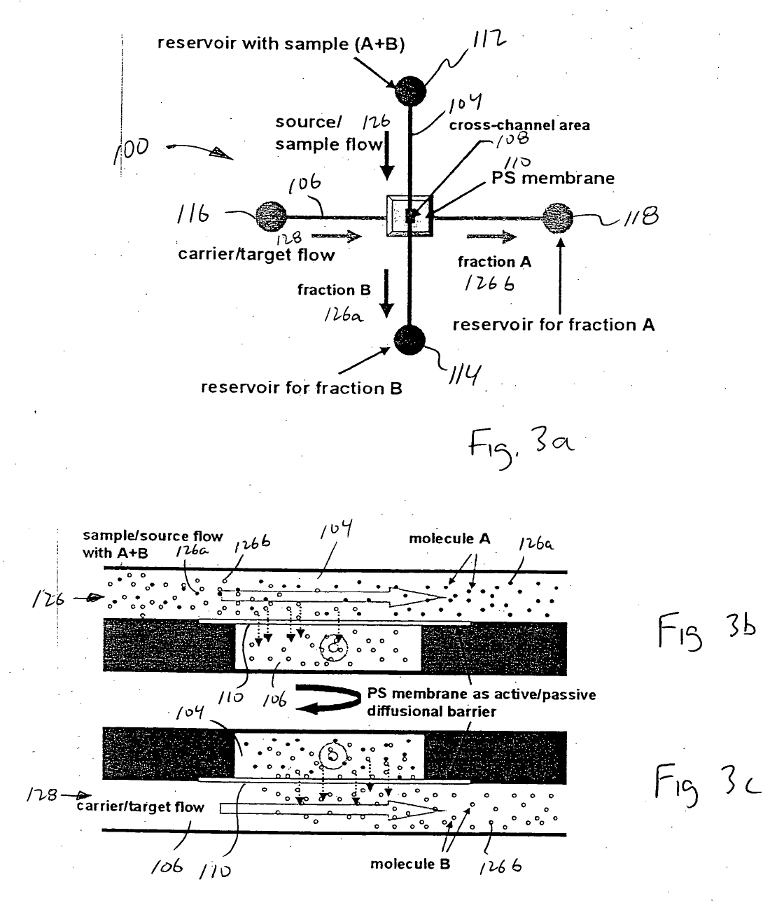

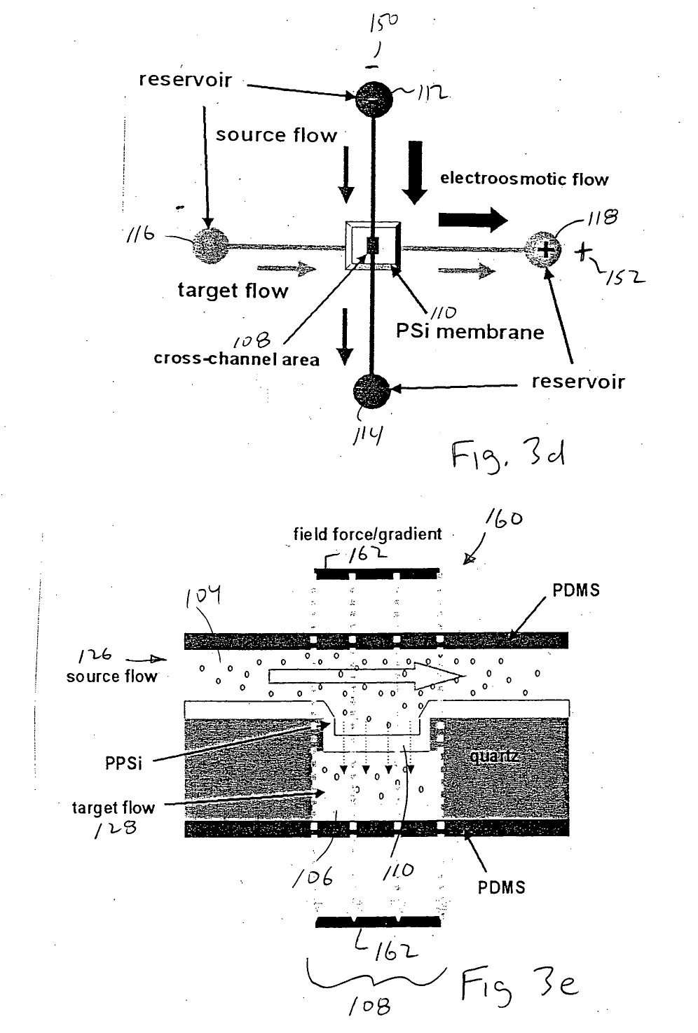

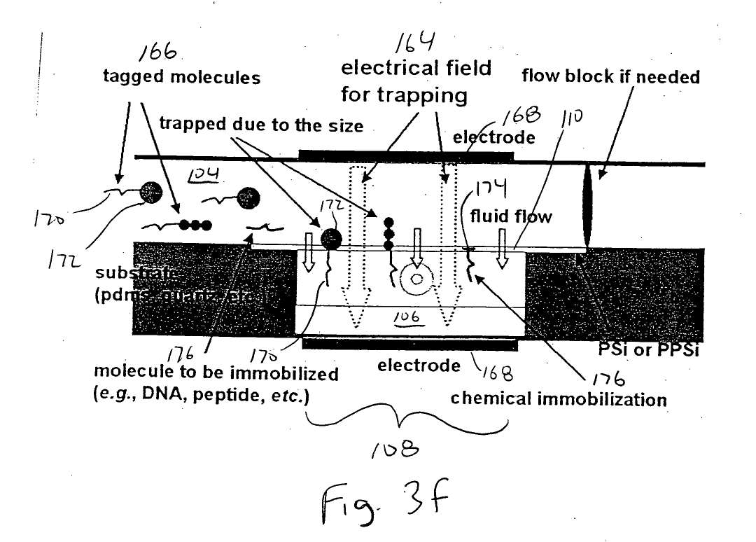

The microfluidic device 100 also includes an electrical portion that may be used in the fractionation, separation or trapping of molecules in the cross-channel area 108 of the device. FIGS. 3a-c show how fluid would normally flow through the microfluidic device 100. FIG. 3a is a plan view, FIG. 3b is a sectional side view of the upper channel 104 in the cross-channel area 108 and FIG. 3c is a sectional view of the lower channel 106 in the cross-sectional area 108 (FIG. 3c is rotated 90 degrees from FIG. 3b). A source fluid sample 126, containing two molecules 126a and 126b, enters the upper microfluidic channel 104 at the input reservoir input reservoir 112 and flows toward the cross-channel area 108. A carrier fluid sample 128 enters the lower channel 106 at the input reservoir 116 and flows toward the cross-channel area 108. At the cross-channel area 108, a portion of the sample fluid 126b will flow through or attach to the porous membrane 110, causing a reaction, such as a potential change in an optical and/or electrical characteristic of the porous membrane 110. Such a characteristic change may be measured in the manners described below.

FIG. 3d shows one embodiment in which an electric field is applied to the microfluidic device 100 for electrokinetic and electroosmotic manipulation of molecules and fluids. A negative charge (−) 150 is applied to the input reservoir 112 of the upper channel 104 and a positive charge (+) 152 is applied to the output reservoir 118 of the lower channel 106. Electroosmotic fluid movement is generated from the upper channel 104, through the porous membrane 110, into the lower channel 106, as indicated by the solid arrows 154.

As disclosed above, in certain embodiments an electric field may be used to apply a voltage, sometimes a high voltage, to the device. Bigger fluidic channels and larger flow volumes require higher voltages to drive electrophoretic and electroosmotic microfluidic flows. In typical microfluidic structures, higher voltages will drive fluids (with molecules) faster/stronger. However, higher voltages may also tend to generate gas bubbles at the interface between fluid and surface of the fluidic channels, causing fluidic transport problems. The charging of the reservoir is shown for convenience, and charging can be applied to any part of the channel. In general, the voltage difference between two applied points create an electrical field, covering a whole fluidic conduit, and applying the voltage to two reservoirs at two opposite ends will cover a whole continuous channel. Normally, high voltage will not affect the PSi membrane very much, and structurally the voltages partitioned over the thickness (distance) of the PSi membrane compared to the voltage applied between two reservoirs is very small. In general “constant” field force/gradient should have “constant” effects on PSi, if any. The electrical field can work on any fluids with ionizable species, such as water, water with salts and/or any charged or ionized molecules. Typically, 10V-1000 V are used depending on the fluidic sizes/structures for milliliters of fluids. Typical microfluidic MEMS devices that handle pico/nano/micro-liters of fluids only need 10 mV-50V because of the smaller sizes/structures.

FIG. 3e shows one embodiment of the microfluidic device 100 used as a molecular fractionator with a field force/gradient mechanism 160 applied near the cross-channel area 108 and is used to influence, change or modify the fractionation process. The field force/gradient mechanism 160 may also be used as a sensor element or as part of a sensor. The field force/gradient may an electric field, magnetic field, acoustic wave, ultrasounds, light with specific wavelengths and other fields capable of interacting with the molecules of interest. In FIG. 3e, an electric field is shown proximate the cross-channel area 108. The electric field is generated using electrodes 162 located on both sides of the cross-channel area 108. The electrodes 160 may be applied externally or may be integrated into the microfluidic device 100 fabrication process.

FIG. 3f shows another embodiment of the microfluidic device 100 that is used as a microfluidic bioreactor with a molecular trapping mechanism 164, using the porous membrane 110 along with electrokinetic (EK) mechanisms as a micro-scale platform for (bio) chemical reactions, including biochemical synthesis, enzymatic reactions, (bio) chemical modifications of macromolecules, colloids, particles, bimolecules, such as DNA, RNA, peptides and proteins, and their complex, for further processing and/or analysis. An electric field is used to create electrophoretic and dielectricphoretic trapping and/or control and particularly can jiggle/vibrate a molecule to let them go through the nanopores of the porous membrane 110 faster and thermodynamically more favorable. The molecules are tapped in the porous membrane 110 due to their attached large “tags” or chemical immobilization. After the (bio) reactions in the microfluidic bioreactor, the molecules can be analyzed in an integrated micro-flow cell or diverted into other microfluidic channels for further processing and/or external assay/analysis, such as DNA sequencing and peptide or DNA detection. Another advantage of using the porous membrane to trap and/or immobilize molecules and filter out (i.e., sieving) solution containing molecules is that the trapped molecules in the solution are properly oriented, trapped and controlled by EK electric field. Pieces of the disclosed embodiments herein may be integrated with other embodiments as a structure for a “lab-on-a-chip” (microfluidics, trapping, bioreactors, filtering, etc.).

The embodiment of FIG. 3f enables the microfluidic bioreactor to perform (bio) reactions for a small number of molecules, including a “single molecule”, in conjunction with other single molecule level disperser or microfluidic separator using a small reaction flow cell/chamber volume/size of the device. The device may combine passive trapping (e.g., filtering, sieving, sizing, etc.) in addition to active molecular trapping and/or control by EK, and at the same time operate as a bioreactor, and in integrated structure for “lab-on-a-chip” bioreactor.

The molecular trap 164 uses an electric field generated by electrodes 168 on both sides of the cross-channel area 108. The electrodes 168 may be applied externally or may be integrated into the molecular fractionator 100 fabrication process. The tagged molecules 166 consist of a molecule 170 with an attached tag 172. The tags 172 are larger than the pore size of the porous membrane 110 are designed to be trapped in the porous membrane 110. The molecule 170 is able to go through the porous membrane 110 while the tag 172 is caught. The electric field can be used to control the movement of the molecules 170. In other embodiments, a molecule 176 may be trapped by chemical immobilization. The porous membrane 110 may be treated with a chemical 174 that binds to the molecule 176, such as with ligand coupling, as it flows through, as shown in the figure. The non-trapped portion of the molecules may also be processed for bioreactions such as modifications of oligo-nucleotide attached to tags (biomolecular nanotags, metallic nanotags, plastic/polymer nanotags, etc.) or cleavage of a tag from a molecule for further processing. For example, one end of a DNA molecule may be trapped/immobilized in the device through chemical or attached tags. The DNA molecule can then be processed or modified such as cleaving one base at a time by exonuclease for DNA sequencing, cleaving at a certain sequence for specific DNA re-sizing, and modifications, ligations, etc.

Electrical fields, such as gravitational/centrifugal, acoustic, magnetic, etc., are field force/gradients, which are modulators of the mobility of the sample/analyte molecules in a fluid where “modulation” means influencing such as facilitating or inhibiting/disturbing the speed/rate of the flowing molecules, driven by microfluidic transport methods such as electrokinetic (e.g., electrophoretic, dielectrophoretic, electroosmotic, etc.), magnetohydrodynamic, hydrodynamic, etc. The “influencing” here means both positive and negative speed changes as well as totally “trap” or “stop” the molecules as well. Because different molecules (with different charges, hydrophobicity/hydrophilicity, shapes/configurations, mass, etc.) are modulated differently by the same modulating field force/gradient, the different molecules are separated. In general “constant” field force/gradient should have “constant” effects on PSi, if any. Each different kind of field works on the corresponding molecules such as electrical field for charges molecules, magnetic field for magnetic molecules, gravitational field for different masses, etc. As described above for electrical fields, different molecules, sizes, structures require a broad ranges of values typically such as 10 mV-1000V for electrical fields and 1 mT-1000 mT for magnetic fields.

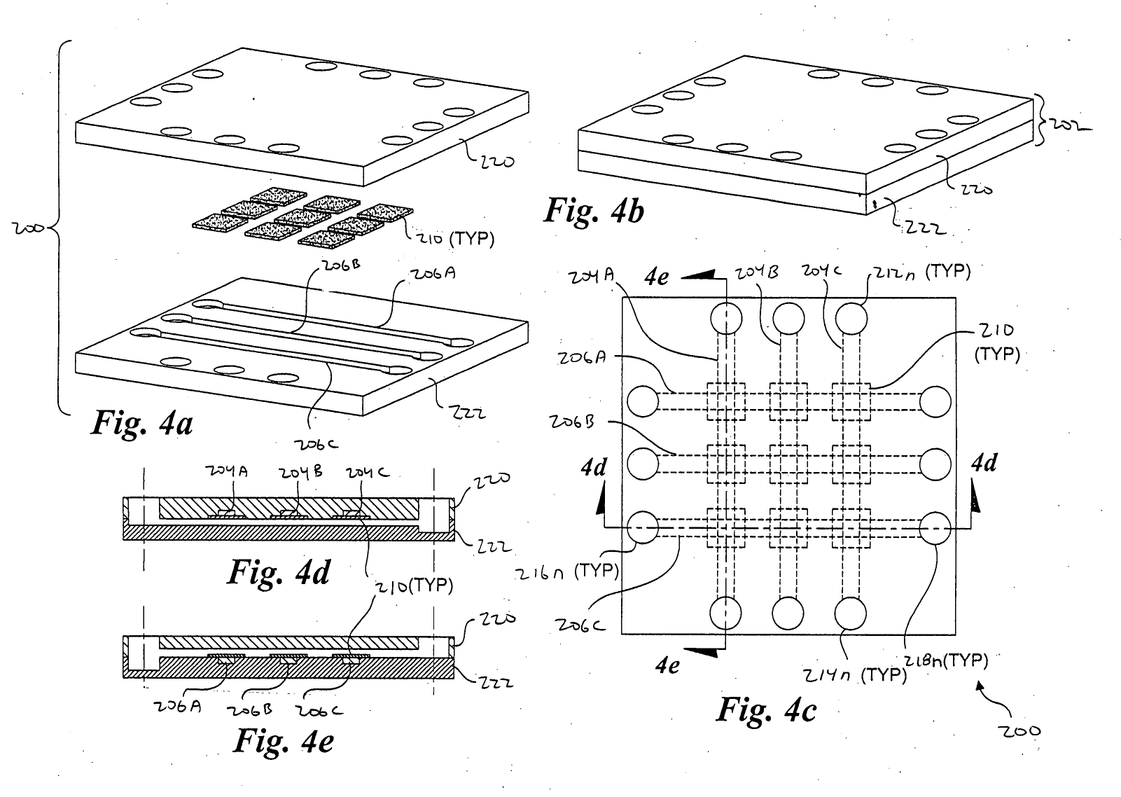

A microfluidic device 200 in accordance with another embodiment of the invention is shown in FIGS. 4a-e. Microfluidic device 200 includes a substrate in which a plurality of upper channels 204A, 204B, and 204C formed in an upper substrate member 220 and a plurality of lower microfluidic channels 206A, 206B, and 206C formed in a lower substrate member 222. Optionally, a plurality of input reservoirs 212n (a-c) and 216n and output reservoirs 214n and 218n may also be provided. In one embodiment, a plurality of porous membranes 210 are disposed within respective recesses (not shown) in upper substrate member 220 in a manner similar to that described above for FIG. 2.

FIG. 5 shows the fluid flow through the microfluidic device 200, similar to microfluidic device 100 shown in FIGS. 3a and 3b. A source fluid sample 226a-c, containing two molecules, enters the upper microfluidic channels 204a-c at the input reservoir 212a-c and flows toward the cross-channel area 208. A carrier fluid sample 228a-c enters the lower channel 106 at the input reservoirs 216a-c and flows toward the cross-channel areas 208. At the cross-channel areas 208, a portion of the sample fluid 226 will flow through or attach to the porous membrane 210, causing a reaction, such as a potential change in an optical and/or electrical characteristic of the porous membrane 210. Such a characteristic change may be measured in the manners described below.

Real-Time Detection Of Biological and Chemical Molecules/Compounds

Various embodiments the porous membrane 110, 210 may be manufactured such that it may be used as a sensor in addition to its filtering/sieving/separation/trapping capability. For example, the porous membrane may be manufactured to produce a changed optical and/or electrical characteristic in response to being exposed to a targeted fluid or reaction, either through use of the base substrate material (e.g., PSi or PPSi), or through the addition of a sensor layer or through chemical doping and the like. Generally, such PSi or PPSi sensor mechanisms may include but are not limited to optical interferometric reflectivity, capacitance modulation, photoluminescence, optical form birefringence, acoustic, etc.

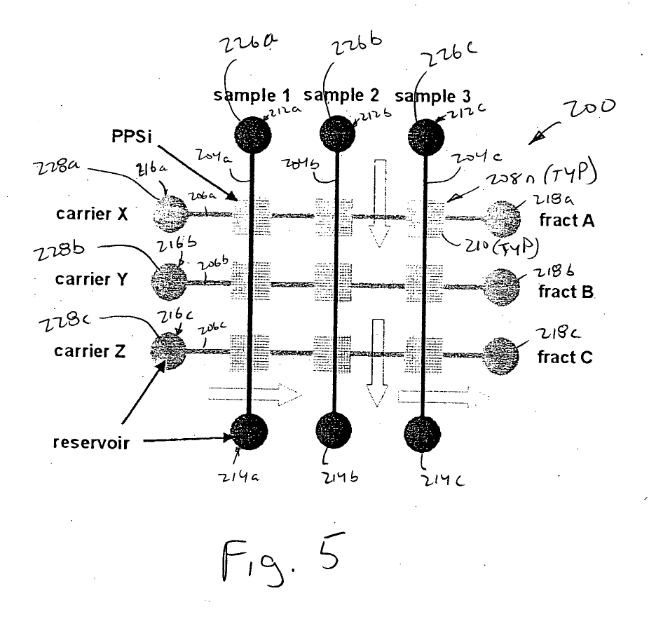

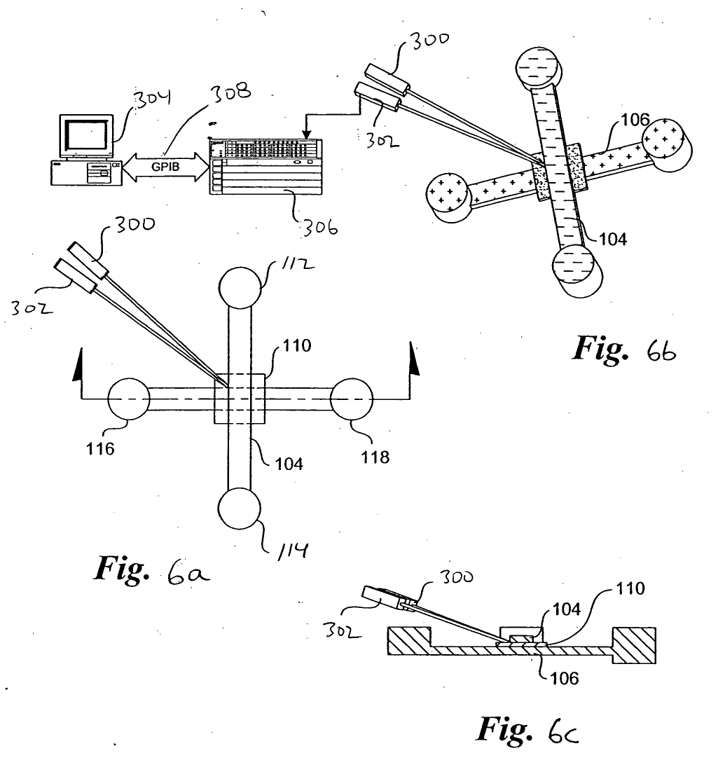

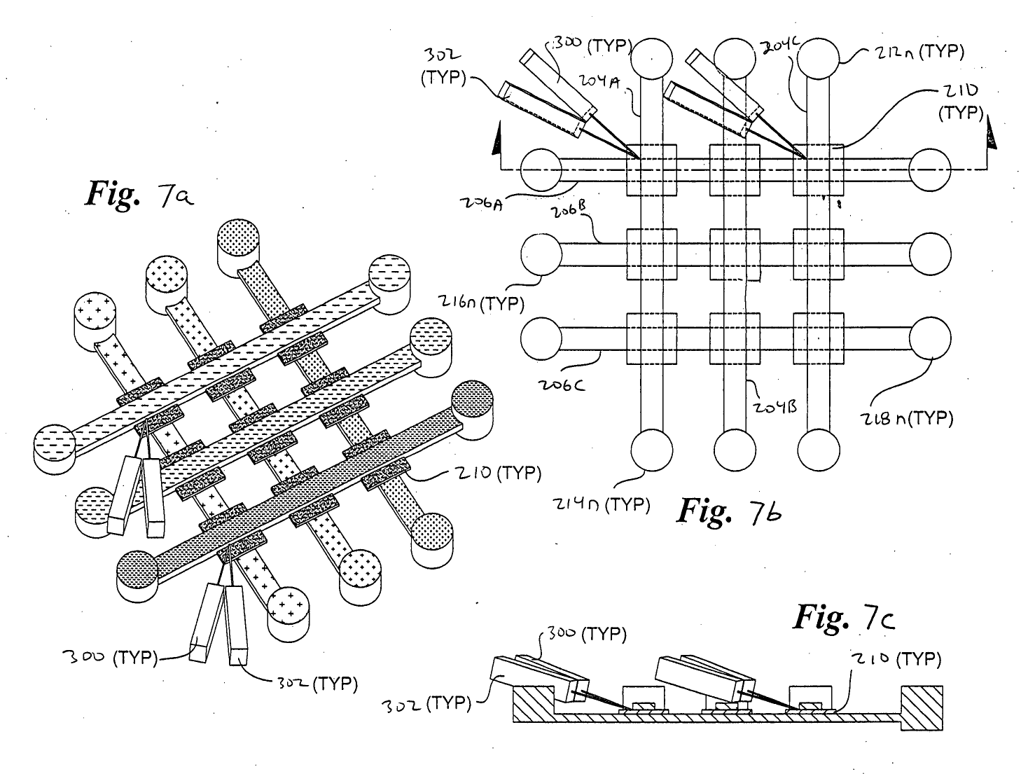

In one embodiment, optical changes may be observed by means of light source 300 and optical detector 302, as shown in FIGS. 6a-c and 7a-c. (It is noted in these Figures only the volumes occupied by the reactant fluids, also commonly referred to solutes and analytes, are shown for clarity). Furthermore, the sizes of the various components are not drawn to scale for clarity. Additionally, the dashes and crosses represent different chemical or biological compounds used for the reactions, wherein different cross-hatch densities and patterns depict different compounds). In general, the light source 300 may comprise any device that produces light suitable for detecting a change in a light characteristic of the porous membrane/sensor 110, 210 in combination with corresponding optical detection equipment or devices 302. For example, in one embodiment the light source 300 comprises a laser source that produces light at a specific wavelength.

Depending on the particular optical characteristics of the porous membrane/sensor 110, 210, visible or invisible light may be used. For visible light wavelengths, at least one of the upper and lower substrates should be visibly transparent, meaning the substrate(s) produces minimal attenuation of visible light. In some instances, it may be desirable to use light having a wavelength in the non-visible spectrum (infra-red or ultra-violet). The light emitted and/or scattered may be detected such as absorption, luminescence (fluorescence and phosphorescence), vibrational (infra-red, Raman, resonance Raman, etc.), SPR (surface plasmon resonance), etc. with or without the use of any “surface enhancements” on PSi membranes using integrated metals and/or chemical functionalization. Many substrate materials are “optically translucent” to these wavelengths, meaning these materials enable light having certain non-visible wavelengths to pass through with minimal attenuation. As an option, various viewing hole configurations may be defined in substrates that are opaque to light having a wavelength that may be used to detect the change in the optical characteristic of the porous membrane (not shown).

Generally, a variety of optical detectors 302 may be employed, depending on the particular optical characteristic to be observed. In one embodiment, the optical detector 302 comprises a detector suitable for laser interferometry. Other typical optical detectors include, but are not limited to, avalanche photodiodes, various photosensors, and other devices used to measure wavelength, phase shift, and or optical energy/power.

Typically, the optical detector 302 may either include build-in data logging facilities, or external data logging equipment may be connected to the optical detector, such as depicted by a data logger 306. As another option, a computer 304 with a data-logging card or an electronic instrument interface, such as the GPIB (General Purpose Instrumentation Bus) interface 308 may be used. The data logger 306 may store the data locally, or on a computer network, such as in a data store hosted by a database or data system or storage area network (SAN) device.

For changes in an electrical characteristic, various electronic instrumentation and/or circuits may be electrically coupled to the porous membrane/sensor 110, 210 to sense the changed condition. This may be facilitated by microelectrical traces disposed in the substrate 102, 202, such as depicted by microelectronic traces 400 in FIG. 8. Optionally, the substrate 102, 202 may be directly wired to external circuitry and/or electrical equipment, such as via wire bonding and the like. In one embodiment, signal conditioning and/or test measurement circuitry may be fabricated directly on or in the platform substrate 102, 202, as is common in the semiconductor manufacturing arts, as depicted by integrated circuit 402. The integrated circuit 402 may either include a build-in electronic measurement device, or an electronic measurement equipment may be connected to the integrated circuit 402, such as depicted by an electronic measurement device 406. As another option, a computer 404 with an electronic instrument interface, such as the GPIB (General Purpose Instrumentation Bus) interface 408 may be used. The electronic measurement device 406 may store the data locally, or on a computer network, such as in a data store hosted by a database or data system or storage area network (SAN) device.

Porous Membrane Manufacture and Characteristics

In accordance with one aspect, the porous membrane comprises a porous structure that may be used for filtering, metering, separating, trapping chemical and/or biological molecules. In general, a porous membrane may be manufactured such that its porosity is greatest along a selected direction. Furthermore, through the manufacturing process described below, the pore sizes can be tuned from a few nanometers to micrometers, thereby enabling the filtration, metering and separation of targeted chemical and biological molecules.

In general, the porous membranes and porous membrane/sensors may be made from a wide-range of materials in which nano- and micro-porous structures may be formed. For example, such materials include, but are not limited to, single crystal porous silicon (PSi), porous polysilicon (PPSi), porous silica, zeolites, photoresists, porous crystals/aggregates, etc. Typically, the porous membranes will be used for molecular separation and/or molecular (bio)reaction media with built-in real-time detection/monitoring of processes, molecules, fluids, reaction states, etc.

In one embodiment, porous silicon is used for the porous membrane. Porous silicon is a well-characterized material produced through galvanostatic, chemical, or photochemical etching procedures in the presence of HF (hydrofluoric acid). Porous silicon can be made generally as complex, anisotropic nanocrystalline structure in silicon layers by either electrochemical etching or stain etching to form porous silicon. The size and orientation of the pores can be controlled by the etching conditions (e.g., current density, etc.) and substrate type and its electrochemical properties. Typical pore sizes range from ˜50 angstrom to ˜10 μm with high aspect ration (˜250) pores in silicon maintained over a distance of several millimeters.

As discuss above, the porous membrane may be made fabricated as an integral part of the substrate. One or more of the substrate layers may be etched, either by electrochemical etching or stain etching, to form porous silicon (PSi). The porosity, pore size, orientation of the pores, etc, are controlled by the etching conditions (e.g., current, density, etc.) and substrate type and its electrochemical properties.

Another type of porous silicon can be formed by spark erosion resulting in a silicon surface with pits and hills of various sizes in the micrometer to nanometers scale. Silicon nanostructures can be produced by an anisotopic etch followed by oxidation. Through oxidizing a microcrystalline film deposited by chemical vapor deposition, silicon crystallites are passivated by SiO to form nanocrystalline structures.

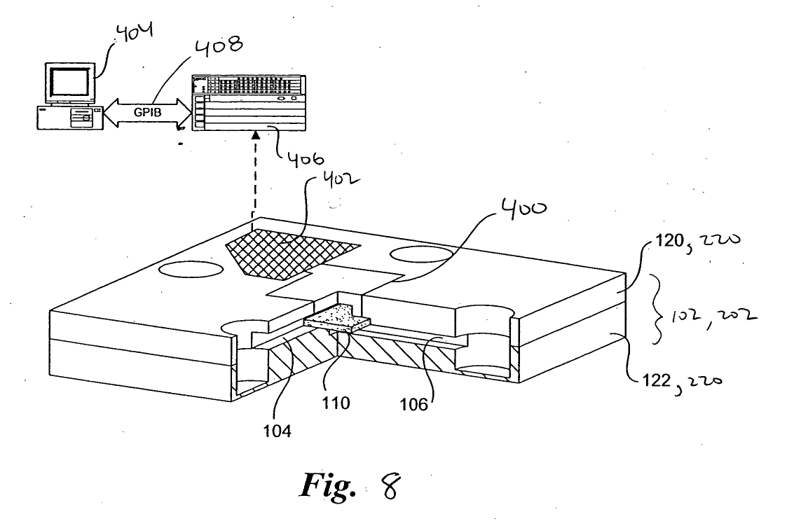

With reference to the flowchart of FIG. 9a, a process for manufacturing porous membrane 110, 210 in accordance with one embodiment of the invention proceeds as follows. First, in a block 500, porous silicon is etched in a silicon layer of typically ˜0.01-50 μm thickness either electrochemically or by stain etching to form porous silicon. In another embodiment, porous polysilicon (PPSi) is deposited by low-pressure chemical vapor deposition (LPCVD), in accordance with a block 502. The size and orientation of the pores, porosity, grain size, thickness, etc., may be controlled via appropriate etching conditions (e.g., current density, current duration, etc.), deposition conditions (e.g., temperature, pressure, etc.), and also substrate type and its electrochemical properties, etc.

Next, in a block 504, a porous silicon (PSi) film (or porous polysilicon (PPSi) film) is physically separated by electropolishing “lift-off” from the PSi-etched or PPSi-deposited silicon and suspended in solution. Alternately, PPSi film may be formed when directly deposited on a substrate (e.g., silicon, quartz, etc.), and can be physically separated by any of various standard etching or micromachining techniques. The PSi or PPSi film is then secured within a corresponding recess formed in a substrate half proximate to a cross-channel area in a block 706.

In an alternate process shown in FIG. 9b, porous polysilicon (PPSi) is directly deposited over the substrate cavity using low pressure chemical vapor deposition (LPCVD) to from the porous membrane in a block 600. Subsequently, in a block 602 a channel is etched in the substrate having a portion that passes under the deposited PPSi. Generally, the substrate may comprise any suitable material in which the microfluidic channels may be formed (e.g., silicon, quartz, polydimethyl siloxane (PDMS), photoresists, and polymers such as polymethylmethacrylate (PMMA), etc.)

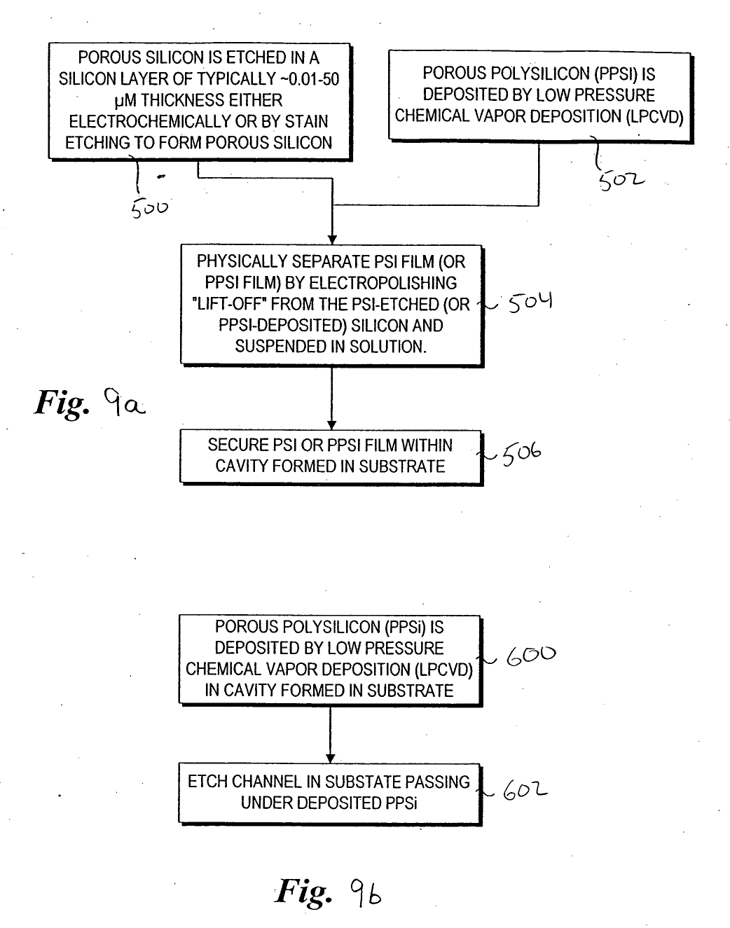

FIG. 10 shows one embodiment of the fabrication steps of a microfluidic device 700. A quartz wafer 702 (substrate) is provided. A hardmask 712 is deposited on a surface of the quartz wafer 702 and patterned 714 forming a cavity or first trench 716. A polysilicon is deposited over the substrate cavity 716 forming a layer of polysilicon 718 using low pressure chemical vapor deposition (LPCVD) at appropriate conditions (e.g., temperature, pressure, etc.). From the other side of the quartz wafer 702, a second trench 720 is created by sawing 722 with a diamond dicing saw blade in an appropriate angle to the substrate cavity or first trench 716. Typically, the angle between the trenches is 90 degrees. The cavity of the second trench 720 is then expanded by chemically etching 724 (such as HF) until the quartz layer is removed so the only the polysilicon or porous membrane 710 layer separates the first trench 716 from the second trench 720. Upper and lower layers 726, 728 of polydimethyl siloxane (PDMS) are attached by standard bonding attachment methods to form an upper microfluidic channel 704 and a lower microfluidic channels 706.

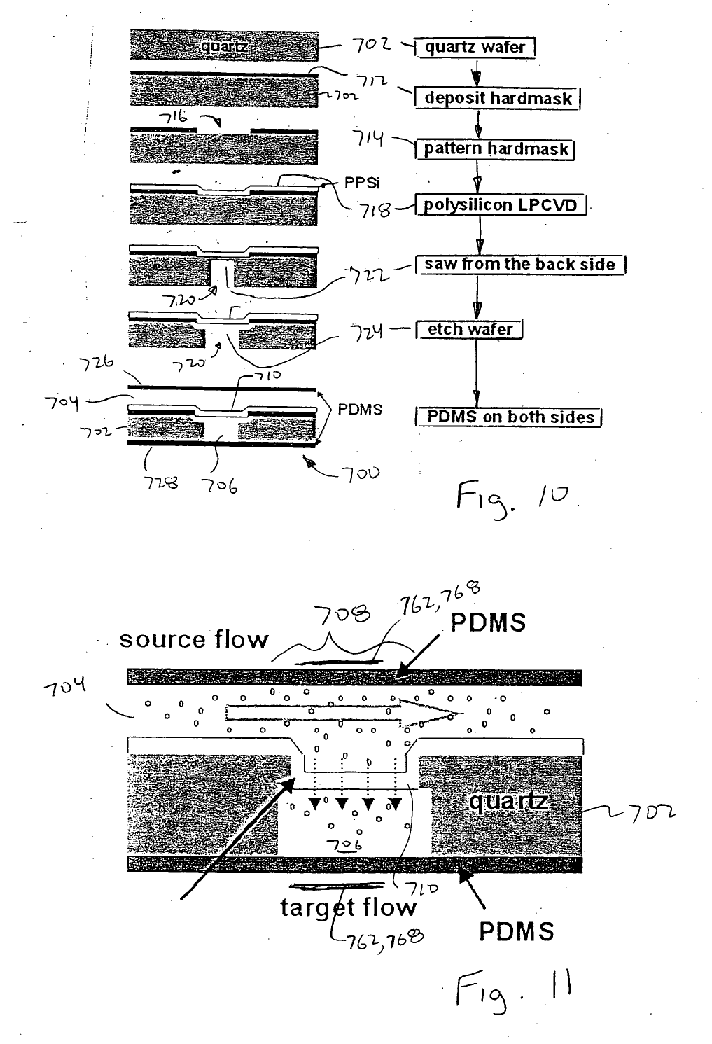

FIG. 11 shows the cross-channel area 708 and the fluid flow in the microfluidic device 700. The microfluidic device 700 includes an upper channel 704 and a lower channel 706 separated by a porous membrane 710. Electrodes 762, 768 may be positioned on each side of the porous membrane 710.

Generally, the size of the channels and the cross-channel reactant area occupied by the porous membrane may be adjusted for the various reactants used in the testing. The flow of the fluids and molecules can be generated by standard microfluidics methods such as hydrostatic pressure, hydrodynamic, electrokinetic, electroosmotic, hydromagnetic, acoustic and ultrasound, mechanical, electrical field induced, heat-induced and other know methods. The flow-through micro-channel configurations allow flow-rate control, fluid dilutions, effective wash-out of the channels, minimum back-flow. Optionally, the flow may be blocked for incubations, diffusions, dilutions, etc., using standard microfluidic components and devices.

Furthermore, massively parallel configurations in accordance with the principles illustrated by the embodiments of FIGS. 4a-e and 5 may be manufactured and employed for testing. In such instance, the porous membrane at each cross channel may have the same or different functionality (optical, biochemical, electrical, acoustic, etc.) as a sensor/detector, molecular separation or sieving filter, bioreactor (with surface modified nanopores, nanopores with immobilized biomolecules, surface coated nanopores, etc.)

While the invention is described and illustrated here in the context of a limited number of embodiments, the invention may be embodied in many forms without departing from the spirit of the essential characteristics of the invention. The illustrated and described embodiments, including what is described in the abstract of the disclosure, are therefore to be considered in all respects as illustrative and not restrictive. The scope of the invention is indicated by the appended claims rather than by the foregoing description, and all changes that come within the meaning and range of equivalency of the claims are intended to be embraced therein.

1. A microfluidic device, comprising:
a source fluid flow channel;
a target fluid flow channel, the target fluid flow channel being in fluid communication with the source fluid flow channel at a cross-channel area;
a porous membrane separating the source fluid flow channel from the target fluid flow channel in the cross-channel area; and
a field-force/gradient mechanism proximate the porous membrane.

2. The device of claim 1, wherein the field-force/gradient mechanism may include an electric field, a magnetic field, an acoustic wave, ultrasound or light with a specific wavelength.

3. The device of claim 1, further comprising a molecular trapping mechanism for trapping one or more tagged molecules.

4. The device of claim 3, wherein the molecular trapping mechanism includes a nanopore membrane with pores capable of trapping the tagged molecules due to their tags.

5. The device of claim 4, wherein the pores are between 50 angstroms and 10 micrometers.

6. The device of claim 3, wherein the molecular trapping mechanism includes a chemically treated portion of the porous membrane in which the tagged molecules are immobilized in the porous membrane through (bio)chemical immobilization or ligand coupling between the chemically treated portion and the tag.

7. The device of claim 3, wherein the molecular trapping mechanism is part of the field-force/gradient mechanism and includes an electric field generator proximate the porous membrane capable of electrophoretic or dielectrophoretic trapping and control of the tagged molecule.

8. The device of claim 1, further comprising a sensor.

9. The device of claim 8, wherein the porous membrane is the sensor.

10. The device of claim 1, further comprising a light source and a detector, the light source and the detector being focused at the cross-channel area.

11. The device of claim 1, wherein the thickness of the porous membrane is between 0.01 and 50 micrometers.

12. The device of claim 1, wherein the porous membrane is capable of fractionating molecules based on size, molecular weight, charges, chemical affinity or other chemical/physical properties.

13. The device of claim 1, wherein the porous membrane is made of a single crystal porous silicon (PSi).

14. The device of claim 1, wherein the porous membrane is made of a porous polysilicon (PPSi).

15. The device of claim 1, further comprising a substrate, the source fluid flow channel and the target fluid flow channel being formed in the substrate.

16. The device of claim 15, wherein the substrate is made of polydimethyl siloxane (PDMS).

17. The device of claim 15, wherein the substrate is made of silicon.

18. The device of claim 15, wherein the porous membrane is integral with the substrate.

19. The device of claim 1, wherein the device is a disposable device.

20. The device of claim 1, wherein the device is a reusable device.

21. The device of claim 1, wherein the source fluid flow channel and the target fluid flow channel intersect at a 90 degree angle at the cross-channel area.

22. A microfluidic molecular-flow fractionator device, comprising:
a substrate, the substrate including:
one or more source fluid flow channels;
one or more target fluid flow channels in fluid communication with the one or more source fluid flow channels; and
one or more cross-channel areas at the intersection of each source fluid flow channel and each target fluid flow channel;
a porous membrane positioned in each cross-channel area separating the source fluid flow channels from the target fluid flow channels; and
a field-force/gradient mechanism proximate the porous membrane.

23. The device of claim 22, wherein the field-force/gradient mechanism may include an electric field, a magnetic field, an acoustic wave, ultrasound or light with a specific wavelength.

24. The device of claim 22, further comprising a molecular trapping mechanism for trapping one or more tagged molecules.

25. The device of claim 24, wherein the molecular trapping mechanism includes a nanopore membrane with pores capable of trapping the tagged molecules due to their tags.

26. The device of claim 25, wherein the pores are between 50 angstroms and 10 micrometers.

27. The device of claim 24, wherein the molecular trapping mechanism includes a chemically treated portion of the porous membrane in which the tagged molecules are immobilized in the porous membrane through (bio)chemical immobilization or ligand coupling between the chemically treated portion and the tag.

28. The device of claim 24, wherein the molecular trapping mechanism is part of the field-force/gradient mechanism and includes an electric field generator proximate the porous membrane capable of electrophoretic or dielectrophoretic trapping and control of the tagged molecule.

29. The device of claim 22, further comprising a sensor.

30. The device of claim 29, wherein the porous membrane is the sensor.

31. The device of claim 22, further comprising a light source and a detector, the light source and the detector being focused at the cross-channel area.

32. The device of claim 22, wherein the thickness of the one or more porous membranes are between 0.01 and 50 micrometers.

33. The device of claim 22, wherein the one or more porous membranes are capable of fractionating molecules based on size, molecular weight, charges, chemical affinity, or other chemical/physical properties.

34. The device of claim 22, wherein the one or more porous membranes are made of a single crystal porous silicon (PSi).

35. The device of claim 22, wherein the one or more porous membranes are made of a porous polysilicon (PPSi).

36. The device of claim 22, wherein the substrate is made of silicon.

37. The device of claim 22, wherein the substrate is made of polydimethyl siloxane (PDMS).

38. The device of claim 22, wherein the one or more porous membranes are integral with the substrate.

39. The device of claim 22, wherein the device is a disposable device.

40. The device of claim 22, wherein the device is a reusable device.

41. A microfluidic bioreactor device with molecular trapping for trapping tagged molecules, comprising:
a substrate, the substrate including:
one or more source fluid flow channels;
one or more target fluid flow channels in fluid communication with the one or more source fluid flow channels; and
one or more cross-channel areas at the intersection of each source fluid flow channel and each target fluid flow channel;
a porous membrane positioned in each cross-channel area separating the source fluid flow channels from the target fluid flow channels; and
a molecular trapping mechanism for trapping one or more tagged molecules at one or more cross-channel areas.

42. The device of claim 41, wherein the molecular trapping mechanism includes a nanopore membrane with pores capable of trapping the tagged molecules due to their tags.

43. The device of claim 42, wherein the pores are between 50 angstroms and 10 micrometers.

44. The device of claim 41, wherein the molecular trapping mechanism includes a chemically treated semi-permeable porous membrane in which the tagged molecules are immobilized in the porous membrane through (bio)chemical immobilization or ligand coupling.

45. The device of claim 41, wherein the molecular trapping mechanism includes an electric field generator proximate the porous membrane capable of electrophoretic or dielectrophoretic trapping and control of the tagged molecule.

46. The device of claim 41, further comprising a sensor.

47. The device of claim 46, wherein the porous membrane is the sensor.

48. The device of claim 41, further comprising a light source and a detector, the light source and the detector being focused at the cross-channel area.

49. The device of claim 41, further comprising a field-force/gradient mechanism proximate the porous membrane.

50. The device of claim 49, wherein the field-force/gradient mechanism may include an electric field, a magnetic field, an acoustic wave, ultrasound or light with a specific wavelength.

51. A method of fabricating a microfluidic device, comprising:
providing a substrate;
forming a source fluid flow channel on a first side of the substrate;
depositing polysilicon on the substrate and in the source fluid flow channel using low pressure chemical vapor deposition (LPCVD) forming a porous membrane;
forming a target fluid flow channel on a second side, the target fluid flow channel being separated from the source fluid flow channel by the porous membrane; and
positioning a field-force/gradient mechanism proximate the semi-permeable porous membrane

52. The method of claim 51, wherein forming the source fluid flow channel on the substrate includes etching a trench in the substrate.

53. The method of claim 51, wherein forming the target fluid flow channel on a second side of the substrate includes:
sawing a trench from the second side of the substrate near the semi-permeable porous membrane forming the target fluid flow channel; and
chemically etching the trench so that the target fluid flow channel contacts the semi-permeable porous membrane.

54. The method of claim 51, further comprising focusing a light source and a detector at the semi-permeable porous membrane.
